(19) United States
(12) Patent Application Publication
(10) Pub. No.: US 2009/0157158 A1
(43) Pub. Date: Jun. 18, 2009

(54) Self-expanding biodegradable stent

(76) Inventors: Vitezslav Ondracek, Bukovina (CZ); Jaroslava Kufrova, Hradoe Kralove (CZ)

Correspondence Address: LITMAN LAW OFFICES, LTD., POST OFFICE BOX 15035, CRYSTAL CITY STATION, ARLINGTON, VA, 22215-0035, US

(21) Appl. No.: 12/292,141

(22) Filed: Nov. 12, 2008

(30) Foreign Application Priority Data
Dec. 13, 2007 (CZ) 2007-879

(51) Int. Cl. A61F 2/86

(52) U.S. Cl. 623/1.2

(57) ABSTRACT

The self-expanding biodegradable stent is a compressible, resilient mesh stent, which is compressed during delivery to a biological vessel or channel, and which expands to the contours of the vessel or channel upon delivery. The self-expanding biodegradable stent includes a substantially cylindrical main body portion having slightly flared, longitudinally opposed first and second open ends. The substantially cylindrical main body portion is hollow and is formed from an open mesh material, preferably formed as a unitary body from a biodegradable monofilament, such as a polydioxanone monofilament fiber. In order to reduce the possibility of trauma to the interior of the vessel, the open ends are blunted, with end points of the mesh forming a plurality of loops being about each of the first and second open ends, and opposing ends of the filament are interleaved with and bonded to a medial portion of the cylinder.

### CROSS-REFERENCE TO RELATED APPLICATIONS

This application claims priority to Czech Republic utility model patent application number 2007-879, filed Dec. 13, 2007.

### BACKGROUND OF THE INVENTION

1. Field of the Invention

The present invention relates to medical implants, and particularly to a self-expanding biodegradable stent that is compressible for insertion into an organ of the body and that expands after insertion to stay in place by resilience of the stent.

2. Description of the Related Art

Stents (i.e., medical devices that secure patency of tubular organs and vessels) are commonly used in medical practice. If a stent is used for palliative treatment of a malignant stenosis so that removal of the stent from the patient's body is not anticipated, then no special demands are made upon the stent.

However, benign stenoses and the like also indicate the usage of stents. Stents may also be used for treating dehiscences in surgical anastomoses in the gastrointestinal tract, or even for stopping bleeding from esophageal varices. In such cases, the stent is intended to be removed at a future time. If the stent is implanted for a period expected to be longer than about one week, then it is “embedded” or ingrown in the tissue. Removal of the stent is associated with a problem. Serious tissue injury may sometimes occur.

When a removable stent is necessary or desirable, the prospect of a degradable or absorbable stent, in which the degradation or disintegration of the stent occurs in a controlled manner, offers an alternative. Such a stent is not intended to be removed from the patient because once its function has been accomplished or the reason for the implant ceases, the stent degrades and gradually passes from the patient's body in a natural way, possibly with the final products of degradation being absorbed, metabolized, or excreted.

Although fully biodegradable materials (e.g., polylactic acid, polyglycolic acid, polyglactin, polydioxanone, polyglyconate, and others) are available, stents made from such materials suffer the disadvantage of having to be expanded by using, e.g., a balloon (see European Patent No. 615,769). In order to make such a stent self-expanding using conventional techniques, it would have to be either: (i) made from a degradable fiber of a large diameter or from a degradable tube of a thick wall, both of which require a delivery catheter of a large diameter, which is in stark contrast to clinical needs from the point of view of safety; or (ii) observing the dimensions of common self-expanding metallic or nondegradable plastic stents, it would have to be reinforced with, e.g., a metallic wire, whereby the prospect of an irremovable biodegradable stent disappears. A non-reinforced stent made by conventional techniques would exert a very poor, insufficient radial force for relieving a stricture and maintaining patency of the tubular organ.

Thus, a self-expanding biodegradable stent solving the aforementioned problems is desired.

### SUMMARY OF THE INVENTION

The self-expanding biodegradable stent is a compressible, mesh stent. The stent is compressed during delivery to a biological vessel or channel and expands to the contours of the vessel or channel upon delivery. The self-expanding biodegradable stent includes a substantially cylindrical main body portion having longitudinally opposed first and second open ends. The ends of the stent are flared slightly, forming a funnel shape. The substantially cylindrical main body portion is hollow and is formed from an open mesh material, preferably formed as a unitary body from a biodegradable monofilament, such as a polydioxanone monofilament fiber. Opposite ends of the fiber are tucked into the mesh in a medial portion of the stent body.

In order to reduce the possibility of trauma to the interior of the vessel, the open ends are blunted, with end points of the mesh forming a plurality of loops at each of the first and second open ends.

These and other features of the present invention will become readily apparent upon further review of the following specification and drawings.

### BRIEF DESCRIPTION OF THE DRAWINGS

FIG. 1 is a perspective view of a self-expanding biodegradable stent according to the present invention, the break and projection lines indicating a middle section of a long stent removed to fit the stent onto the page.

FIG. 2 is a side view of the self-expanding biodegradable stent of FIG. 1.

FIG. 3 is a top view of the self-expanding biodegradable stent of FIGS. 1 and 2.

FIG. 4 is a diagrammatic elevation view showing a step in a method of making the self-expanding biodegradable stent of FIGS. 1-3.

FIG. 5 is a front view of a loop of the self-expanding biodegradable stent of FIG. 1-3.

FIG. 6 is a side view of the loop of FIG. 5.

Similar reference characters denote corresponding features consistently throughout the attached drawings.

### DETAILED DESCRIPTION OF THE PREFERRED EMBODIMENTS

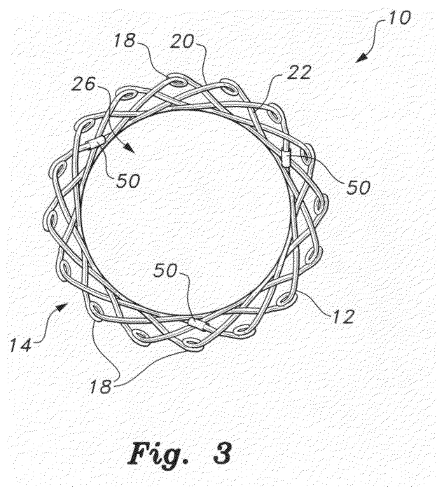

The self-expanding biodegradable stent 10 is preferably formed from a single strand of resilient, biodegradable material, such as a polydioxanone monofilament fiber 12. As best shown in FIGS. 1, 2 and 3, the longitudinally opposed ends 14, 16 of stent 10 are flared and include a plurality of loops 18 formed from fiber 12. The loops 18 form an atraumatic, blunt surface to prevent trauma or damage to tissue when the stent 10 is inserted into a patient, rather than having the mesh form a plurality of sharp end points, as in conventional mesh stents. As best shown in FIG. 3, sixteen such loops 18 are formed about each end, although it should be understood that these sixteen loops 18 are shown for exemplary purposes only, and that any suitable number of loops 18 may be provided, depending upon the diameter and use of the stent 10.

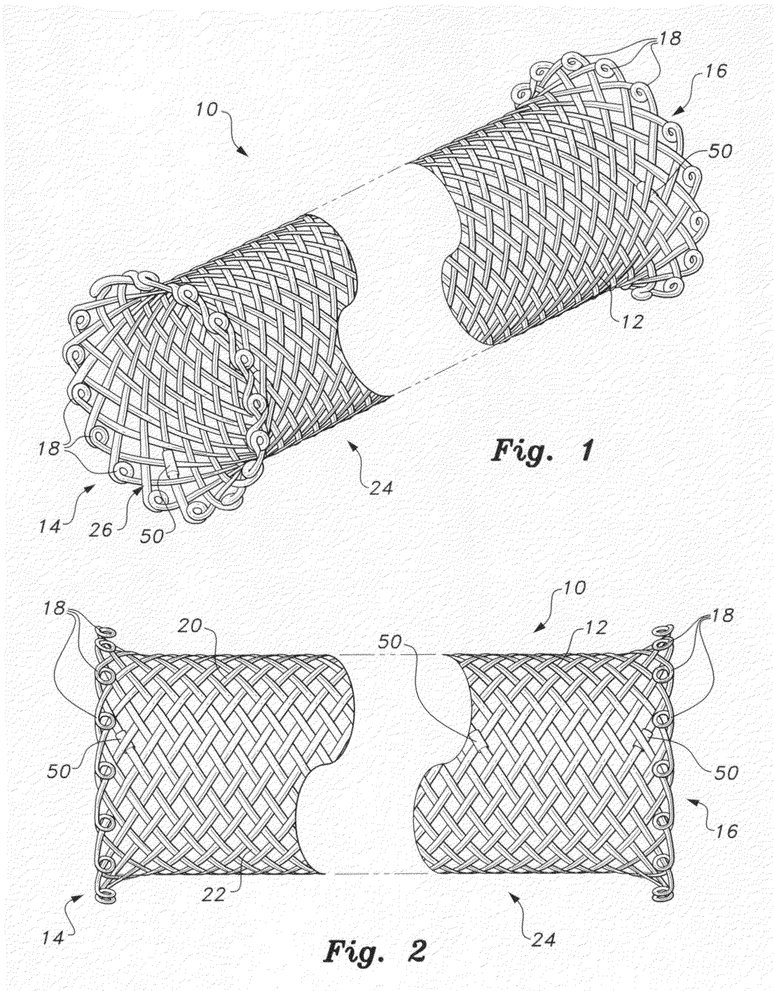

As shown in FIGS. 1 and 2, the stent 10 is formed having a substantially cylindrical central portion 24, with longitudinally opposed flared ends 14, 16. As noted above, each end 14, 16 includes a plurality of loops 18 about the periphery. The stent 10 is preferably formed from a single strand of fiber, and the loops 18 are also formed from this single strand, in a manner that will be described in detail below. The stent 10 defines a hollow, interior region 26 therein. The central portion 24 is formed as a regular mesh. When viewed from above (see FIG. 3), the mesh is formed from a first strand portion 20 forming a helix extending in a counterclockwise direction, and a second strand portion 22 forming a helix extending in a clockwise direction.

Due to the mesh structure and the composition of fiber 12, the stent 10 is compressible. During implantation, the stent 10 is compressed within a catheter and inserted into the desired tubular vessel or channel. Once released, the stent 10 expands both longitudinally and radially, to spread to the dimensions of the vessel or channel. The stent 10 is formed from a biodegradable material, such as polydioxanone, allowing the stent 10 to dissolve within the patient's body over time and then be metabolized, excreted, and possibly partially absorbed. Fiber 12 may further alternatively be coated with an additional biodegradable material.

The biodegradable stent 10 is formed with dimensions that correspond to conventional, nondegradable metallic and plastic stents. The desired mechanical properties are achieved by choice of proper material and proper heat treatment.

In use, the stent 10 is implanted using a conventional delivery catheter having a diameter suitable for implanting a corresponding nondegradable stent. The stent 10 is compressed, both longitudinally and radially, implanted in the tubular organ or vessel, released from the delivery catheter, whereupon the stent 10 spontaneously expands longitudinally and radially, and the delivery catheter is removed. After some time, the stent 10 degrades. For example, a gastrointestinal stent degrades due to the impact of the tissue, food, enzymes, and digestive fluids in the gastrointestinal tract. Metabolism of the polydioxanone fiber produces water and carbon dioxide when carried through to completion. Degradation produces small pieces or debris that may be excreted, or when metabolism is fully carried out, the water and carbon dioxide may either be excreted or absorbed.

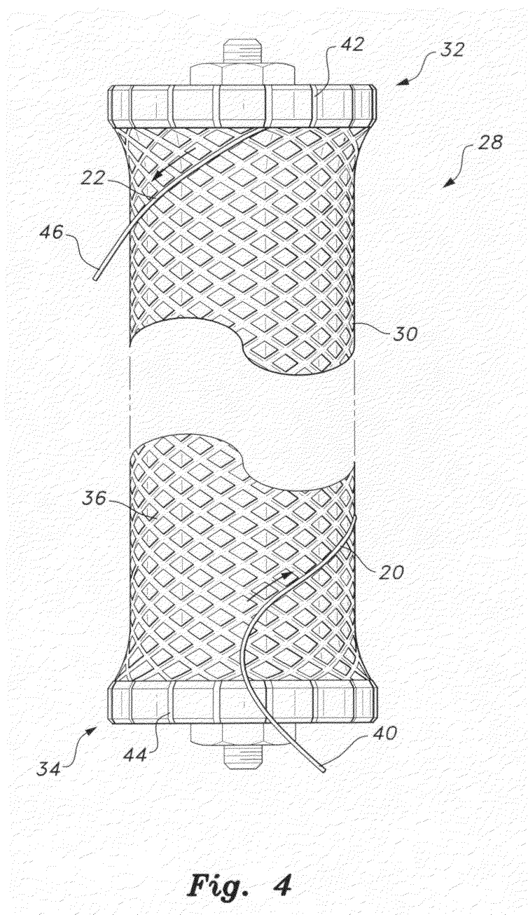

FIG. 4 illustrates a mandrel 28 about which fiber 12 is wrapped in order to weave the stent 10. The mandrel 28 is shaped like the stent 10; i.e., including opposed, flared ends 32, 34, and a central, cylindrical portion 30. Grooves 36 are formed about the outer surface of mandrel 28, as shown, with the grooves 36 forming a mesh pattern corresponding to the mesh of the stent 10.

In order to form the stent 10, a first end 40 of fiber 12 is first fixed to the mandrel 28 at a substantially central position in the central portion 30. In FIG. 4, first end 40 is shown as being free, but it should be understood that this is shown only for purposes of clarification. First end 40 is positioned at any suitable location along central portion 30 during the braiding process. In FIG. 4, end 32 of mandrel 28 corresponds to end 14 of stent 10, thus the fiber 12 extends from end 40 upwardly (in the orientation of FIG. 4), wrapping helically in the counterclockwise direction, forming first strand portion 20.

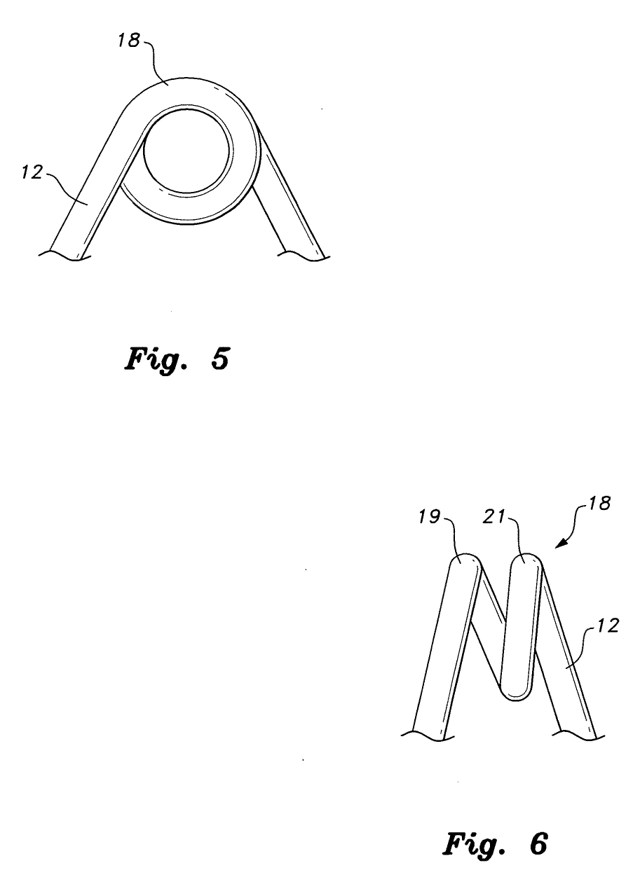

First strand portion 20 extends to upper end 32 of mandrel 28 until it reaches a first pin 42 (preferably formed within one of the plurality of slots or grooves formed on either end, as shown), secured to the upper end 32. The fiber 12 is wound about pin 42 twice, to form a loop 18, and then extends downwardly, wrapping about mandrel 28 helically in the clockwise direction, forming second strand portion 22. FIGS. 5 and 6 illustrate the formation of loop 18, with FIG. 6 showing the double winding of one such loop 18, forming a pair of looped portions 19, 21.

Returning to FIG. 4, the strand is wrapped about mandrel 28 within the grooves 36, as shown. The second strand portion 22 is wound about mandrel 28 until reaching the lower end 34, where it is wrapped around a second pin 44 twice, thus forming a loop 18. The wrapping process is then repeated, with a plurality of pins being formed on both ends 32, 34 to form the closed mesh pattern shown in FIGS. 1-3. The fiber ends 40, 46 are fixed to the mesh in a medial portion of the stent 10 through any suitable bonding process, thus forming a unitary mesh structure, formed from only a single fiber.

Following braiding of strand 12 about mandrel 28, the braided strand and mandrel 28 are heated in a kiln at a constant temperature between 80° C. and 106° C. of approximately 100° C. for a period of approximately 20 minutes. Once the stent 10 has cooled and cured on the mandrel 28, the stent 10 is removed from the mandrel 28.

As shown in FIGS. 1-3, a plurality of radiopaque markers 50 may be attached to the fiber 12 with, preferably, three such markers 50 being shown adjacent each end 14, 16. Each marker 50 is formed as a hollow tube with the fiber 12 passing therethrough, the marker 50 being formed from gold, platinum-iridium alloy, or any other suitable radiopaque material. Preferably, at least one such marker 50 is further fixed to the central portion 24 of stent 10.

It is to be understood that the present invention is not limited to the embodiment described above, but encompasses any and all embodiments within the scope of the following claims.

We claim:

1. A self-expanding biodegradable stent, comprising a substantially cylindrical main body portion having longitudinally opposed first and second open ends, the main body portion being hollow and being formed as an open mesh, a plurality of loops being formed around the periphery of each of the open ends, the open mesh material being resilient in order to compress during implantation and expand to conform to a tubular organ or vessel upon delivery therein.

2. The self-expanding biodegradable stent as recited in claim 1, wherein the open mesh material forming said substantially cylindrical main body portion is formed as a unitary body from a monofilament fiber.

3. The self-expanding biodegradable stent as recited in claim 2, wherein the monofilament fiber is formed from a biodegradable material.

4. The self-expanding biodegradable stent as recited in claim 3, wherein the monofilament fiber is formed from polydioxanone.

5. The self-expanding biodegradable stent as recited in claim 1, wherein each said loop includes a pair of looped portions.

6. The self-expanding biodegradable stent as recited in claim 1, further comprising at least one radiopaque marker attached to said main body portion.

7. The self-expanding biodegradable stent as recited in claim 6, wherein said at least one radiopaque marker comprises a plurality of radio-opaque markers, at least one of the radiopaque markers being attached to said main body portion adjacent each of said first and second ends.

8. The self-expanding biodegradable stent as recited in claim 1, wherein the open mesh material includes first and second fiber portions, each of said first and second fiber portions having a substantially helical shape, the first and second fiber portions being wound in opposite directions.

9. The self-expanding biodegradable stent as recited in claim 1, wherein each of said first and second ends is slightly flared.

10. A method of making a self-expanding biodegradable stent, comprising the steps of:
a) providing a mandrel having a substantially cylindrical main body portion having longitudinally opposed first and second ends, the opposed first and second ends being radially flared, first and second sets of substantially helical grooves being formed in an outer surface of said mandrel, said first set of substantially helical grooves having an opposite chirality from said second set of substantially helical grooves, a plurality of pins being annularly formed about each of said first and second ends;
b) providing a monofilament fiber and securing a first end thereof to a central portion of said mandrel within one of the first set of substantially helical grooves;
c) winding the monofilament fiber about said mandrel within the one of the first set of substantially helical grooves;
d) winding the monofilament fiber about one of said pins formed on the first end of said mandrel to form a loop;
e) winding the monofilament fiber about said mandrel within one of the second set of substantially helical grooves;
f) winding the monofilament fiber about one of said pins formed on the second end of said mandrel to form a loop;
g) winding the monofilament fiber about said mandrel within another one of the first set of substantially helical grooves;
h) repeating said steps d) through g) until the monofilament fiber has been wound about all of said first and second sets of substantially helical grooves and about all of the plurality of pins formed on the first and second ends of said mandrel, resulting in a unitary mesh body;
i) heating the unitary mesh body;
j) curing the unitary mesh body; and
k) removing the unitary mesh body from the mandrel.

11. The method of making a self-expanding biodegradable stent as recited in claim 10, wherein said step i) includes heating the unitary mesh body at a temperature of approximately 100° C. for a time period of approximately 20 minutes.

12. The method of making a self-expanding biodegradable stent as recited in claim 10, wherein said steps of forming loops each include forming a pair of looped portions.

13. The method of making a self-expanding biodegradable stent as recited in claim 10, further comprising the step of securing at least one radiopaque marker to said unitary mesh body.

14. A biodegradable stent, comprising a single filament of polydioxanone fiber helically wound to form an elongated, resilient mesh cylinder having slightly flared ends, the filament being formed into loops at the opposing ends of the cylinder, opposing ends of the filament being interleaved with and bonded to the mesh in a medial portion of the cylinder, the mesh cylinder being heat treated at between 80° C. and 106° C.

15. The biodegradable stent according to claim 14, wherein the helically wound mesh includes both clockwise and counterclockwise turns.
